(19) United States
(12) Patent Application Publication
(10) Pub. No.: US 2013/0046098 A1
(43) Pub. Date: Feb. 21, 2013

(54) KINASE INHIBITORS

(71) Applicants: Lynne Hampson, Manchester (GB); Ian Hampson, Manchester (GB); John Anthony Hadfield, Manchester (GB); Timothy Patrick Kemp, Kearsley Bolton (GB); Alan Thomson McGowan, Manchester (GB)

(72) Inventors: Lynne Hampson, Manchester (GB); Ian Hampson, Manchester (GB); John Anthony Hadfield, Manchester (GB); Timothy Patrick Kemp, Kearsley Bolton (GB); Alan Thomson McGowan, Manchester (GB)

(73) Assignee: THE UNIVERSITY OF MANCHESTER, Manchester (GB)

(21) Appl. No.: 13/392,253

(22) PCT Filed: Aug. 20, 2010
(86) PCT No.: PCT/GB10/51385

§ 371 Date: May 31, 2012

(30) Foreign Application Priority Data
Aug. 24, 2009 (GB) 0914726.5

(51) Int. Cl. C07D 213/75

(52) U.S. Cl. 546/309

(57) ABSTRACT

The present invention relates to compounds of formula (I): wherein A=formula: (a) or formula: (b); X is oxygen or methylene; Y is C₃₋₆ alkyl or aryl; Z is oxygen or C₁₋₃ alkyl; R¹ is hydrogen or C₁₋₃ alkyl; R² is hydrogen or C₁₋₃ alkyl; the or each R³ is separately C₁₋₃ alkyl or halo; the or each R⁴ is separately C₁₋₃ alkyl or halo; p is 0 to 4; q is 0 to 4; m is 0 or 1; and n is 1 to 3. These compounds are useful as kinase inhibitors for the treatment of cancer and other diseases.

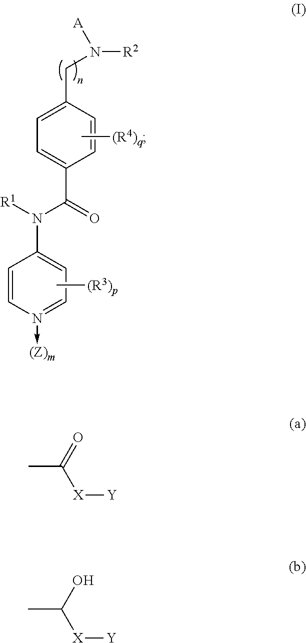

The present invention relates to new chemical compounds that exhibit biological activity which suggests that they have the potential to modulate the activity of selected kinases, and the use of said compounds as kinase inhibitors. The invention also relates to these compounds for use as medicaments. Such medicaments may be useful in the prevention and/or treatment of cancer.

Cancer is one of the leading causes of human mortality, being implicated in around an eighth of all human deaths, a proportion that rises in developed countries. In light of the widespread mortality associated with cancer there remains an unmet need for treatment regimes and medicaments of use in the prevention and/or treatment of cancer.

It is known that the development and progression of cancer may be associated with over activity of cellular kinases. For example, the activation of RhoA kinase (ROCK) is well known to be involved in the process of tumour cell invasion. It has also been suggested that kinases, such as ROCK, may play a role in malignant transformation associated with the development of cancer.

In light of the above, it will be appreciated that kinase inhibitors represent a promising class of compounds for use in the prevention and/or treatment of cancer. Accordingly, there is a need for the development of new compounds capable of inhibiting kinase activity. While kinase inhibitors may be of particular interest in the prevention and/or treatment of cancer, there are a range of other illnesses or diseases, including polycystic kidney disease and conditions associated with pain or inflammation, in which they may also have utility.

The object of the present invention is to obviate or mitigate one or more of the above problems.

According to a first aspect of the present invention there is provided a compound having the formula (I)

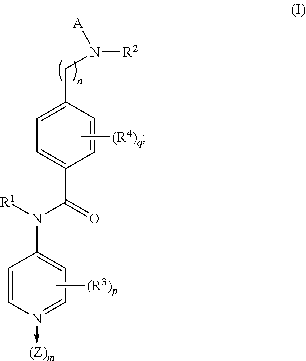

wherein:

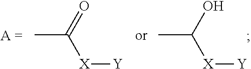

X is oxygen or methylene;
Y is C₃₋₆ alkyl or aryl;
Z is oxygen or C₁₋₃ alkyl;
R¹ is hydrogen or C₁₋₃ alkyl;
R² is hydrogen or C₁₋₃ alkyl;
the or each R³ is separately C₁₋₃ alkyl or halo (that is, where p is 2, 3 or 4, each R³ substituent may be the same or different, for example, if p is 2, then one R³ group may be C₁₋₃ alkyl and the other R³ group may be a halo group);
the or each R⁴ is separately C₁₋₃ alkyl or halo (that is, where q is 2, 3 or 4, each R⁴ substituent may be the same or different);
p is an integer from 0 to 4;
q is an integer from 0 to 4;
m is 0 or 1; and
n is an integer from 1 to 3.

Compounds according to formula (I) have demonstrated the ability to inhibit the growth of transformed cells. Transformed cell lines represent an in vitro model of cancer cells, and the ability of a compound of interest to inhibit transformed cells in vitro provides a good indication that the compound in question may be used for the prevention and/or treatment of cancer in vivo.

The compounds of the invention inhibit the activity of a number of kinases, including, but not limited to, RhoA dependent RhoA kinases (also referred to as “ROCKs”) ROCK1 and ROCK 2; p38 (also referred to as MAP4K4); Hgk (also referred to as MAPK14); and Aurora A (also referred to as AURKA). Without wishing to be bound by any hypothesis, the inventors believe that the ability of the compounds of the invention to inhibit kinase activity contribute to their inhibitory activity in respect of transformed cells.

Where the term “alkyl” or “alkyl group” is used herein without any further qualification it is to be interpreted as encompassing both substituted and unsubstituted alkyl groups. Moreover, where the term “alkyl” or “alkyl group” is used herein without any further qualification it will be understood to encompass linear, branched and cyclic alkyl groups.

Where the term “aryl” or “aryl group” is used herein without any further qualification it is to be interpreted as encompassing both substituted and unsubstituted aryl groups. Any substitution may be provided as an appendage to the carbocyclic ring structure and/or within the carbocyclic ring structure wherein at least one carbon atom forming part of the aryl ring structure is replaced with a non-carbon atom so as to provide a heteroaryl ring structure, e.g. a pyridinyl group.

In a first preferred embodiment of the first aspect of the present invention there is provided a sub-class of compounds of formula (I) in which A is —C(O)—X—Y and which have the formula (II)

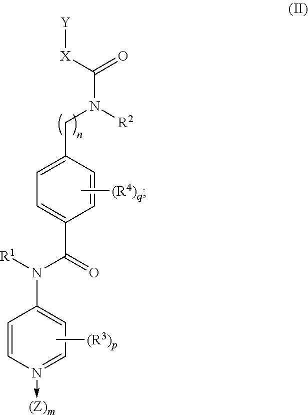

In formulae (I) and (II) it is preferred that the or each R³ is separately C₁₋₃ alkyl, in which the or each alkyl group may be substituted or unsubstituted, linear or branched, saturated or unsaturated as appropriate. Preferred R³ groups are methyl, ethyl, n-propyl and i-propyl. One or more R³ group may be a halo group, most preferably fluoro, but also including chloro, bromo or iodo.

p is an integer from 0 to 4, and may therefore be 0 such that no R³ groups are present and both pairs of ortho- and meta-carbon atoms of the pyridine are ‘unsubstituted’, i.e. bonded to a hydrogen atom. This is the configuration represented in the second and third preferred embodiments of the first aspect of the present invention set out below.

Alternatively, p may be 1 such that one of the ortho- or meta-pyridine ring carbon atoms carries an R³ group rather than a hydrogen atom.

As a further alternative, p may be 2 in which case the two R³ groups may be the same or different, for example, one may be a C₁₋₃ alkyl group (e.g. methyl) while the other may be a halo group (e.g. F). The two R³ groups may both be provided at the ortho-position of the pyridine ring, or one R³ group may be provided at the ortho-position and one at the meta-position.

In a further alternative p is 3, in which case each of the three R³ groups may be the same or different, or two of the R³ groups may be the same and the remaining R³ group may be different. Two of the three R³ groups may be provided at the ortho-position, with the remaining R³ provided at the meta-position; alternatively, two of the three R³ groups may be provided at the meta-position and the remaining R³ group provided at the ortho-position.

In a further preferred alternative p is 4 and both ortho- and meta-carbon atoms of the pyridine ring are substituted with R³ groups. All four R³ groups may be the same, or all four may be different. Three of the R³ groups may be the same and one different. Two of the R³ groups may be the same and the other two may be different, or two of the R³ groups may be the same and the other two R³ groups may be the same as one another, but different to the other R³ groups.

With regard to the R⁴ group(s) in formulae (I) and (II) it is preferred that the or each R⁴ is separately C₁₋₃ alkyl, in which the or each alkyl group may be substituted or unsubstituted, linear or branched. Preferred R⁴ groups are methyl, ethyl, n-propyl and i-propyl. One or more R⁴ group may be a halo group, most preferably fluoro, but also including chloro, bromo or iodo.

In a preferred embodiment where q is 0, no R⁴ groups are present and both pairs of ortho- and meta-carbon atoms of the phenyl ring are ‘unsubstituted’, i.e. bonded to a hydrogen atom, as in the second and third preferred embodiments of the first aspect of the present invention set out below.

Alternatively, q is 1, and one of the ortho- or meta-phenyl ring carbon atoms carries an R⁴ group rather than a hydrogen atom.

As a further alternative, q is 2, in which case the two R⁴ groups may be the same or different, for example, one may be a C₁₋₃ alkyl group (e.g. methyl) while the other may be a halo group (e.g. F). The two R⁴ groups may both be provided at the ortho-position of the phenyl ring, or one R⁴ group may be provided at the ortho-position and one at the meta-position.

In a further alternative where q is 3, each of the three R⁴ groups may be the same or different, or two of the R⁴ groups may be the same and the remaining R⁴ group may be different. Two of the three R⁴ groups may be provided at the ortho-position, with the remaining R⁴ provided at the meta-position; alternatively, two of the three R⁴ groups may be provided at the meta-position and the remaining R⁴ group provided at the ortho-position.

In the further preferred alternative where q is 4, both ortho- and meta-carbon atoms of the phenyl ring are substituted with R⁴ groups. All four R⁴ groups may be the same, or all four may be different. Three of the R⁴ groups may be the same and one different. Two of the R⁴ groups may be the same and the other two may be different, or two of the R⁴ groups may be the same and the other two R⁴ groups may be the same as one another, but different to the other R⁴ groups.

In a second preferred embodiment of the first aspect of the present invention there is provided a sub-class of compounds of formula (II) having the formula (III)

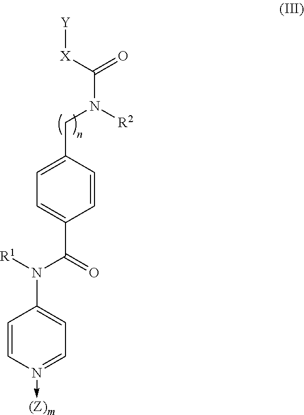

In formulae (I), (II) and (III), R² is preferably hydrogen. Alternatively, R² is a C₁₋₃ alkyl group, wherein the alkyl group may be substituted or unsubstituted, linear or branched, saturated or unsaturated as appropriate. R² is preferably an unsubstituted C₁₋₃ alkyl group. Preferred R² groups are methyl, ethyl, n-propyl and i-propyl, which may be substituted or unsubstituted, but are most preferably unsubstituted.

m may be 0, in which case the pyridine nitrogen atom retains its lone pair of electrons, or m may be 1, in which case the nitrogen lone pair is involved in a dative bond to an oxygen atom to form an N-oxide derivative (in the preferred embodiment where Z is oxygen), or in a dative bond to a C₁₋₃ alkyl group, preferably a methyl group to form an N-methyl charged salt.

As stated above in the first aspect of the present invention, n is an integer from 1 to 3, in which case the phenyl group is spaced from the nitrogen atom of the upper amide group by one, two or three methylene linker groups. In formulae (I), (II) and/or (III), n is preferably 1, as in the third preferred embodiment of the first aspect of the present invention shown below.

In a third preferred embodiment of the first aspect of the present invention there is provided a sub-class of compounds of formula (III) having the formula (IV)

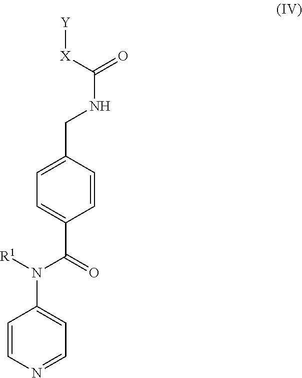

In formulae (I) to (IV), Y is preferably C₃₋₆ alkyl, which may be substituted or unsubstituted, and may be linear, branched or cyclic, optionally including one or more unsaturated group. Optional substituents include halo groups, such as fluoro, chloro, bromo or iodo groups.

Y may be a linear C₃₋₆ alkyl group, such as an n-propyl, n-butyl, n-pentyl, or n-hexyl group.

It is preferred that Y is a relatively bulky group, and so Y is more preferably a branched C₃₋₆ alkyl group, such as an i-propyl, i-butyl, t-butyl, i-pentyl, t-pentyl, i-hexyl or t-hexyl group. A particularly preferred Y group is t-butyl, as used in compounds (V), (VI) and (VII) set out below and which exhibited extremely encouraging biological activity as explained more fully below. A preferred derivative of t-butyl replaces the three methyl groups with one, two or, most preferably, three trifluoromethyl groups such that Y is —C(CF₃)₃.

As a further alternative of a relatively bulky Y group, Y may be a substituted or unsubstituted cyclic C₅₋₆ alkyl group. The cyclic group may be saturated, e.g. cyclopentane or cyclohexane, or unsaturated and include one unsaturated group (carbon-to-carbon double bond) at any desired location, e.g. cyclopentene or cyclohexene, or two unsaturated groups at any desired location, e.g. 1,2-cyclohexadiene, 1,3-cyclohexadiene or 1,4-cyclohexadiene.

Alternatively, Y may be a substituted or unsubstituted aryl group. Preferred aryl groups include phenyl, benzyl, tolyl or xylyl groups. While any appropriate substituent may be provided, it is preferred that the one or more aryl group substituent is a halo group, such as fluoro, or chloro, bromo or iodo.

X is preferably oxygen such that the substituent group bonded at the para-position of the phenyl group relative to the amido group linked to the pyridine ring is a cabamate group.

Alternatively, X is a methylene linker group, which may be substituted or unsubstituted. If substituted, the methylene linker may carry one or two substituents such as further alkyl groups or halo groups.

R¹ is preferably hydrogen. Alternatively, R¹ may be a C₁₋₃ alkyl group, wherein the alkyl group may be substituted or unsubstituted, linear or branched, saturated or unsaturated as appropriate. A preferred R¹ alkyl group is methyl, other options including ethyl, n-propyl and i-propyl.

A second aspect of the present invention provides a compound of formula (V)

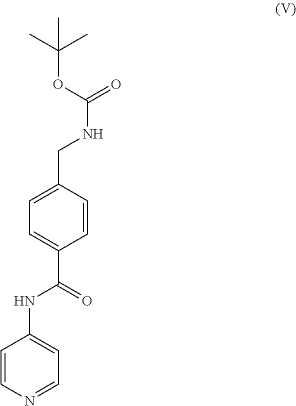 [SMILES: CC(C)(C)OC(=O)NCc1ccc(C(=O)Nc2ccncc2)cc1]

According to a third aspect of the present invention there is provided a compound of formula (VI)

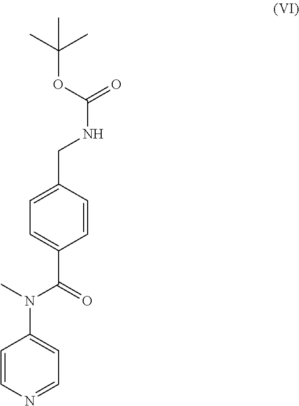 [SMILES: CN(C(=O)c1ccc(CNC(=O)OC(C)(C)C)cc1)c1ccncc1]

A fourth aspect of the present invention provides a compound of formula (VII)

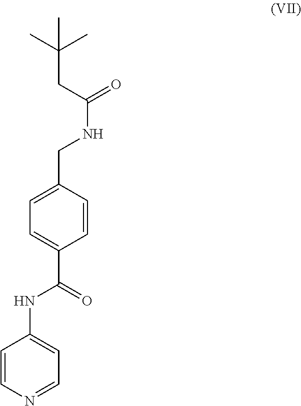 [SMILES: CC(C)(C)CC(=O)NCc1ccc(C(=O)Nc2ccncc2)cc1]

As mentioned above, the compounds of the invention (for present purposes taken to encompass compounds in accordance with the first, second, third or fourth aspects of the invention) exhibit biological activities. These activities include the ability to inhibit kinase activity, and the ability to alter the behaviour of transformed cells. In particular, the compounds of the invention exhibit the ability to inhibit the growth of transformed cells, and to inhibit the formation of transformed cell colonies. Compounds in accordance with the invention (in particular in accordance with formula (V)), are even able to reduce numbers of colonies of transformed cells once such colonies have already formed. Surprisingly, the compounds of the invention appear to be able to achieve these activities even when only transiently provided to such kinases or cells. Thus the biological effects of the compounds of the invention are able to persist even when the compounds are withdrawn.

It will be appreciated that these biological activities of the compounds of the invention are highly suitable to medical uses, and such uses give rise to further aspects of the invention.

In a fifth aspect of the invention there is provided a compound of the invention for use as a medicament. The compound of the invention may be a compound in accordance with the first, second, third or fourth aspects of the invention. Compounds used in accordance with this aspect of the invention may be used as medicaments for use in chemotherapy. Alternatively, or additionally, compounds of the invention may be used in the prevention and/or treatment of diseases such as polycystic kidney disease, and or the prevention and/or treatment of pain or inflammation or of conditions associated with pain or inflammation.

In a sixth aspect of the invention there is provide a compound of the invention for use as a medicament for the prevention and/or treatment of cancer. As before, the compound of the invention may be a compound in accordance with the first, second, third or fourth aspects of the invention. Compounds used in accordance with this aspect of the invention may be used to inhibit the formation and/or growth of metastases.

Without wishing to be bound by any hypothesis, the inventors believe that the compounds of the invention are able to increase incidences of gap junction formation between transformed cells and adjoining non-transformed cells, and that as a result the non-transformed cells are able to inhibit the activity of the transformed cells. This mode of action has not been described before, and is consistent with the inventors finding that the compounds of the invention exert their greatest inhibition of cancer cell activity when the cancer cells are provided in mixed populations with non-transformed cells.

It will be appreciated that, when the compounds of the invention are for use in accordance with the fifth or sixth aspects of the invention, they should be provided in a therapeutically effective amount. A suitable therapeutically effective amount of a compound of the invention may be determined experimentally with reference to considerations such as the identity of the specific compound, the nature of the medical use to which the compound is to be put (e.g. the nature of the condition to be treated, and the progression of the condition within a patient), and the route by which the compound is to be administered.

The compounds for use in accordance with the fifth or sixth aspects of the invention may be formulated with a suitable pharmaceutical excipient. The properties required of a suitable pharmaceutical excipient will be apparent to those skilled in the art with reference to the manner in which a pharmaceutical formulation comprising a compound of the invention is to be used.

The compounds of the invention may be used in the manufacture of medicaments for systemic administration. Alternatively, it may be preferred that the compounds of the invention are used in the manufacture of medicaments for use in localised administration. Such medicaments may be formulated in an appropriate manner for the provision of the compound of the invention to a tissue or organ in which it is desired that the compound exert its biological activity. By way of example, the compounds of the invention may be used in the manufacture of medicaments for localised administration to the skin, or to the cervix.

The finding that the compounds of the invention are able to exert a persistent inhibitory effect on transformed cells, even after direct exposure of the cells to the compound has ceased, indicates that the compounds of the invention may be used in the manufacture of a medicament for use in a discontinuous treatment regime. Thus a preferred embodiment of the fifth or sixth aspects of the invention provides a compound of the invention for use as a medicament in a treatment regime comprising at least one incidence of treatment followed by a period in which no incidences of treatment are administered.

It may be expected that the use of compounds of the invention in a discontinuous treatment regime may be of benefit in reducing side effects otherwise associated with such a treatment regime. For example, the compounds of the invention may be used in a discontinuous chemoprevention or chemotherapy regime. Such a chemoprevention of chemotherapy regime may be of use for the prevention or treatment of cancer.

Another medical use to which the compounds of the invention are suited is the treatment of wound healing. It is known that wound healing and cancer share a number of mechanisms in common, including cell proliferation, extracellular matrix deposition, and tissue remodelling. In a seventh aspect the invention provides the use of the compounds of the invention in the treatment of wounds to prevent fibrosis or scar formation that may otherwise occur as a consequence of wound healing. The compound of the invention may be used to prevent the formation of keloids, which are a form of pathological scarring in which the scar tissue formed grows beyond the boundaries of the initial injury.

Aspects of the present invention will be further described, by way of example only, with reference to the following non-limiting Examples and the accompanying Figures in which:

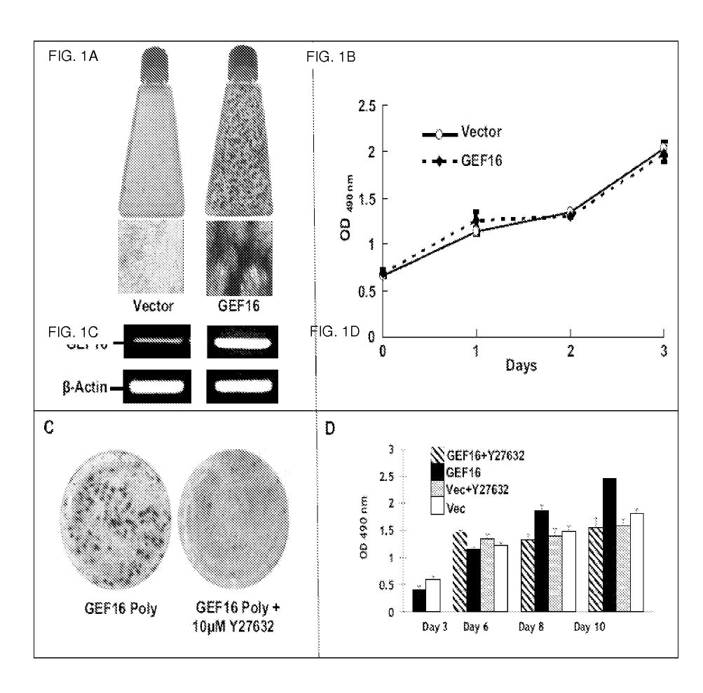

FIG. 1A shows images of GEF16 transformed NIH3T3 colonies and vector control colonies (after 12 days of growth in the presence of G418), where Toluidine blue staining allows visualisation of transformed foci. This figure also shows that GEF16 mRNA expression was verified by RT-PCR;

FIG. 1B shows the results of Cell AQ⁹⁶ growth comparison of Vector and GEF16 polyclonal transfected cells;

FIG. 1C shows photographs of cultures of Polyclonal GEF16 transfected NIH3T3 cells were incubated with either 10 μM of Y27632 or DMSO control for 10 days and stained with Toluidine blue;

FIG. 1D is a bar chart showing the results of Cell AQ⁹⁶ assessment of the growth of GEF16 and vector transfected cells seeded in a 96 well plate at 1×10³ cells per well. These were incubated for 3 days followed by addition of reagent to determine the starting point for the assay. 10 μM of either Y27632 or DMSO were then added to wells containing both cell types and Cell AQ⁹⁶ absorbance measured at 6, 8 and 10 days. At day 3 cells were 100% confluent which was determined by phase contrast visual inspection of the cultures at ×20 magnification;

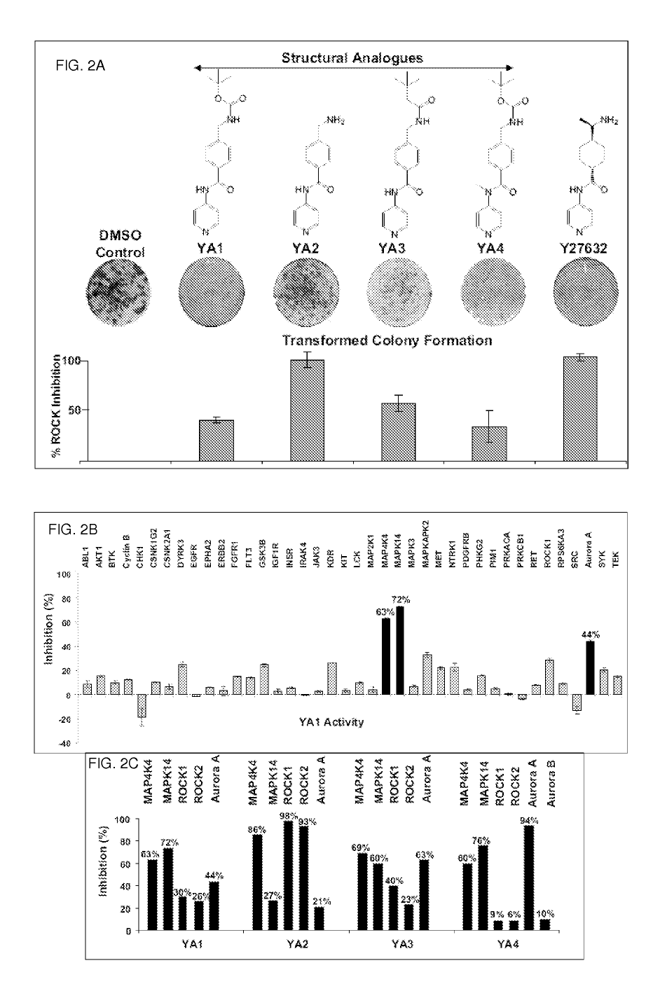

FIG. 2A shows Toluidine blue staining of transformed colonies treated with control, or with Y27632, or with a compound of the invention. Aliquots of 2.0×10⁵ polyclonal GEF16 transfected cells were seeded into 30 mm dishes, incubated over night then treated with 10 μM of 64 different structural analogues of Y27632, including the four compounds of the invention designated YA1, YA2, YA3 and YA4. Colony formation was assayed after 10 days. The ROCK inhibitory activity of the compounds of the invention was also compared to Y27632, and the results of this are shown in the bar chart;

FIG. 2B represents the results of SelectScreen™ assessment of the kinase inhibitory activity of YA1 (a compound of the invention in accordance with formula (V)) in respect of a representative selection of 40 human kinases;

FIG. 2C illustrates single point analysis of the kinase inhibitory activity of the compounds of the invention YA1-YA4 against the kinases: HGK, p38, ROCK 1, ROCK 2 and Aurora A;

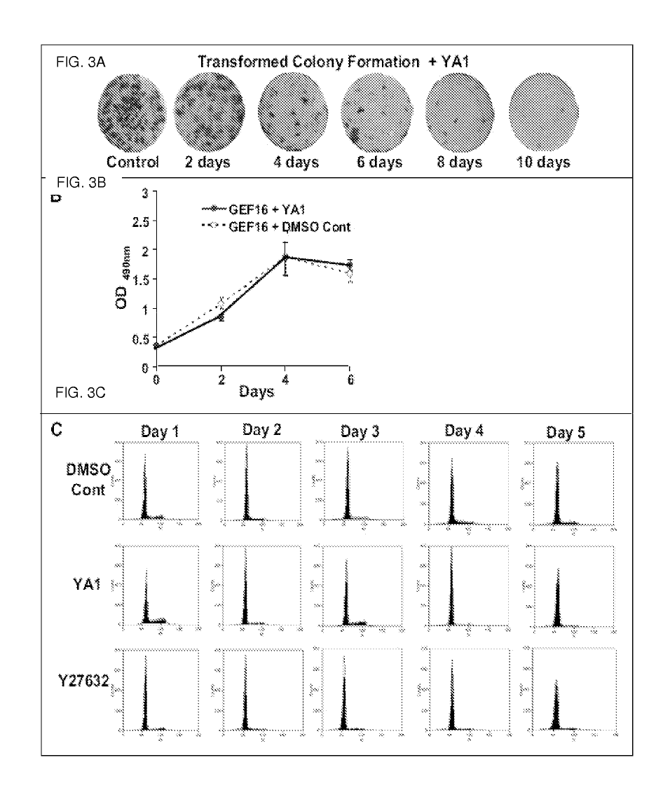

FIG. 3 illustrates that YA1 irreversibly suppresses the formation of GEF16 transformed NIH3T3 colonies. FIG. 3A shows images of polyclonal GEF16 transfected cells (plated at 2.0×10⁵ cells per 30 mm dish) to which 10 μM YA1 has been added for 2, 4, 6, 8, or 10 days respectively. Following this incubation period the compound was then removed from the culture media and cells maintained in normal growth media for a further 10 days before the photographs shown were taken. FIG. 3B illustrates the results of Cell AQ⁹⁶ proliferation assay of sub confluent cultures treated with DMSO or inhibitor for the same time interval. FIG. 3C illustrates flow cytometric analysis of either YA1 or Y27632 (10 μM) treated GEF16 cells.

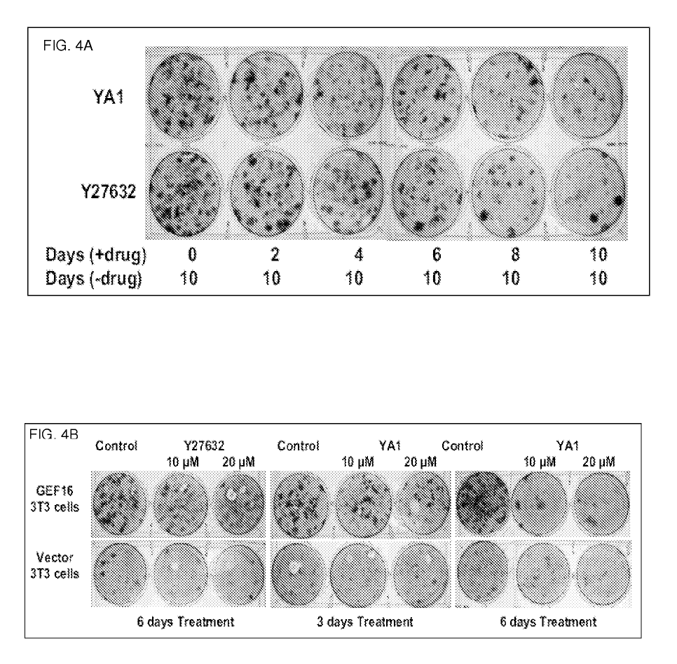

FIG. 4 illustrates results showing that Y27632 and YA1 (a compound of the invention in accordance with formula (V)) irreversibly suppress the formation of GEF16 transformed NIH3T3 colonies. Unlike Y27632, YA1 is also able to eliminate pre-existing transformed colonies. In FIG. 4A polyclonal GEF16 transfected cells were seeded at 2.0×10⁵ cells per 30 mm dish and incubated over night. 10 μM YA1 or Y27632 was then added to each of these for 2, 4, 6, 8, and 10 days respectively whereupon the, cells were detached with trypsin and re-plated at a density of 2.0×10⁵ cells. After a further 10 days culture in the absence of inhibitors the cells were stained with Toluidine blue and photographed to produce the images shown. To produce the results shown in FIG. 4B polyclonal GEF16 and vector transfected cells were seeded at 2.0×10⁵ cells per 30 mm dish and incubated for 10 days after which 10 μM or 20 μM of either, YA1, Y27632 or DMSO control was added. These were incubated for 3 or 6 days then stained with Toluidine blue before being photographed.

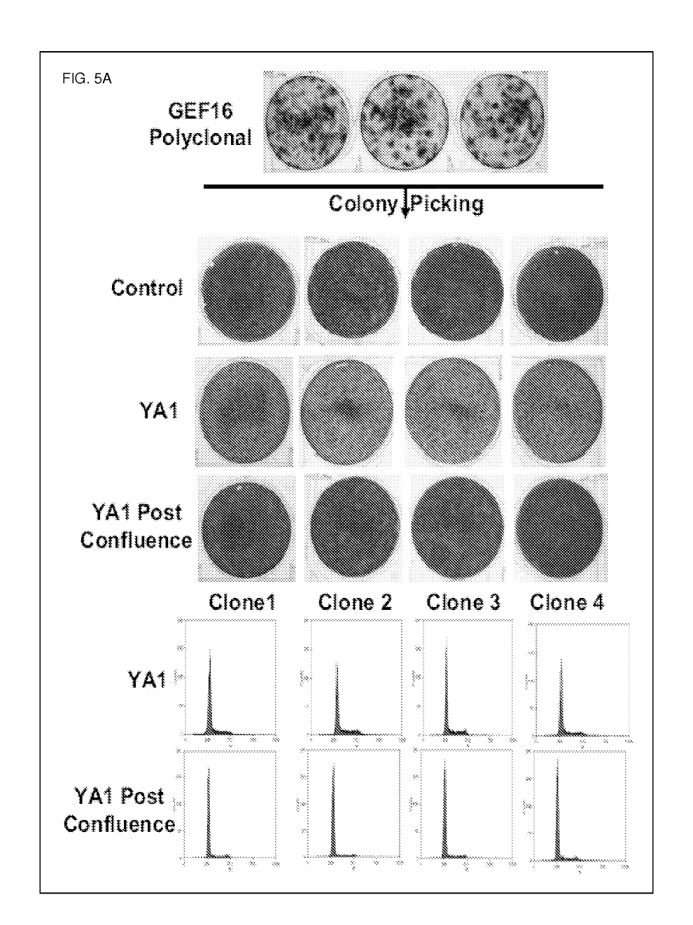

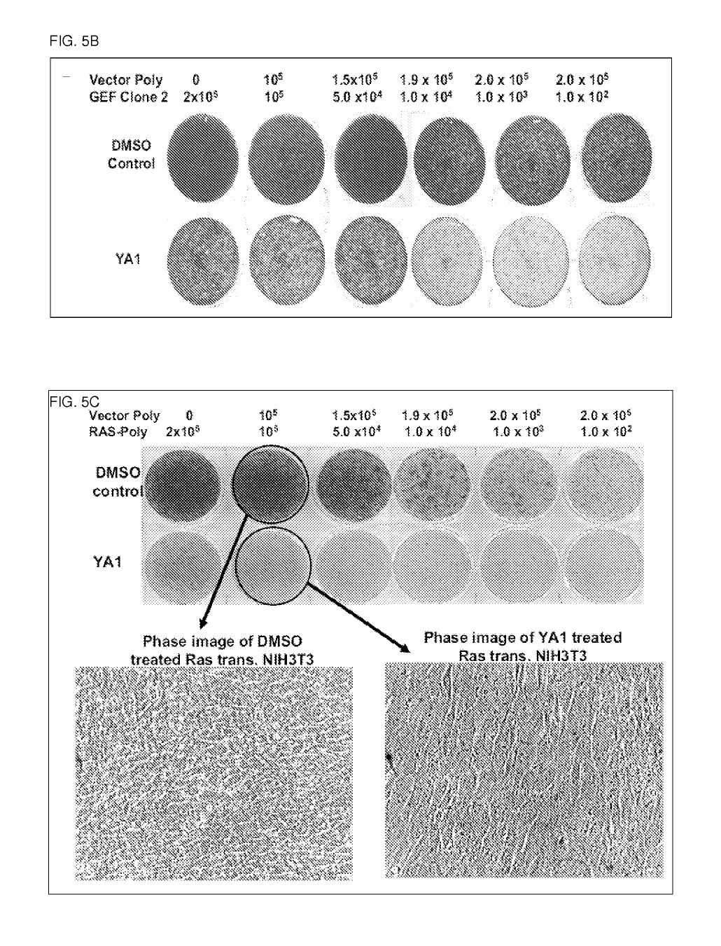

FIG. 5 illustrates that the growth suppressive effects of YA1 on single transformed colony derived GEF16 NIH3T3 cells and polyclonal Ras transformed NIH3T3 cells are more pronounced when these are co-cultured with non-transformed cells. In FIG. 5A single transformed colonies were picked from 10 day cultures of GEF16 polyclonal NIH3T3 cells and expanded. These cells were then seeded at 2.0×10⁵ cells per 30 mm dish and treated with 10 μM of YA1 or DMSO control either immediately or following 10 days in culture. Duplicate wells were harvested for flow cytometry. In FIG. 5B a total of 2×10⁵ cells per well were plated consisting of increasing numbers of non-transformed vector cells co-cultured with decreasing numbers of single transformed colony derived GEF16 cells. These were treated with 10 μM of either YA1 or DMSO for 10 days. In producing the results shown in FIG. 5C the same co-culture experiment described in connection with FIG. 5B was carried out substituting polyclonal Ras transformed NIH3T3 cells for GEF16 transformed cells.

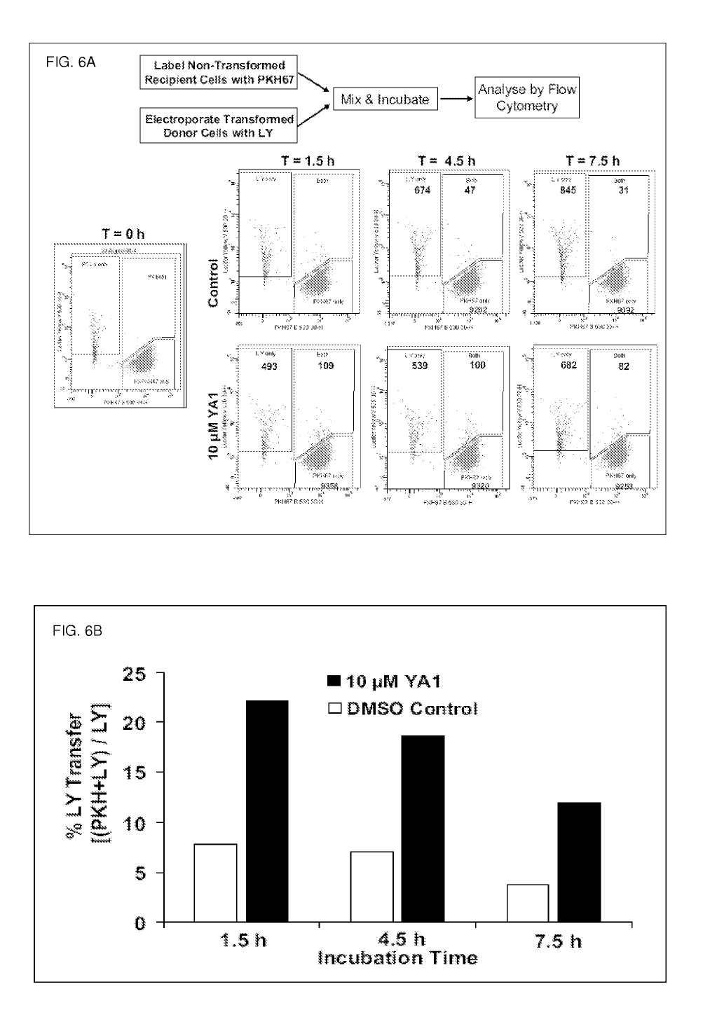

FIG. 6 illustrates that YA1 (a compound of the invention in accordance with formula (V)) stimulates gap junction formation between transformed and non-transformed cells. FIG. 6A shows the results of Study 3 (more fully described below) in which cells derived from a single GEF16 transformed colony were electroporated with LY and co-cultured with non-transformed cells that had been previously labelled with PKH67. Either YA1 (10 μM) or DMSO control was added to duplicate cultures and these harvested at T=0, 1.5, 4.5 and 7.5 hours for analysis by flow cytometry. The numbers of cells displaying each type of fluorescence is shown (“LY only”, “PHK67 only” or “both”). FIG. 6B illustrates the ratio of double LY/PKH67 labelled cells expressed as percentage of the total LY population.

### EXAMPLES

### Study 1 Compounds of the Invention Inhibit Kinase Activity

The ability of the compounds of the invention to act as inhibitors of kinase activity was investigated. Y27632, a structural analogue of the compounds of the invention, is known to inhibit RhoA dependent RhoA kinase (ROCK), and so the ability of compounds of the invention to inhibit this kinase, among others, was studied. Details of the materials and methods used, and the results obtained, are provided below.

### 1.1 Materials and Methods

### 1.1.1 Compounds of the Invention

Four compounds of the invention were manufactured as described elsewhere in the specification. The structures of the four compounds (designated YA1, YA2, YA3 and YA4) are illustrated in FIG. 2. Of these compounds, YA1 is a compound of the invention in accordance with formula (V), YA3 is a compound of the invention in accordance with formula (VII), and YA4 is a compound of the invention in accordance with formula (VI).

### 1.1.1 In Vitro ROCK Activity Assay.

A specific assay was used to evaluate the ability of the compounds of the invention to inhibit ROCK. Rho-kinase activity was determined using an immunoassay as recommended by the manufacturer (Cyclex Co., Ltd., Nagano, Japan). Briefly, 100 μA samples containing 10 mUnit (1 Unit incorporates 1 nmol of phosphate into GST-MBS/MYPT1 per minute at 30° C.) of recombinant ROCK with or without the compounds of the invention were aliquoted into a 96-well plate (100 μl/well), pre-coated with threonine Rho-kinase phosphorylation substrate. After 30 min incubation at 30° C., the plate was washed three times with PBS then incubated with 100 μl/well of HRP conjugated anti-phospho-specific antibody for 1 h at room temperature. The amount of phosphorylated substrate was determined by adding 100 μl/well of substrate reagent for 10 min and the reaction was terminated by adding 100 μl/well of the stop solution. The absorbance was measured on a 96-well plate reader at 450 nm (Dynex Technologies, West Sussex, UK). Each data point was performed in triplicate and the assay was repeated twice.

### 1.1.2 SelectScreen™ In Vitro Kinase Profiling

The SelectScreen™ kinase inhibitor assay service was used (Invitrogen Ltd., Paisley, UK) to investigate the ability of the compounds of the invention to inhibit a large number of different kinases. This commercially provided service allows assessment of the ability of a compound (or compounds) of interest to inhibit a broad panel of human kinases. Further details regarding this service are available on the Invitrogen website at:

www.invitrogen.com/site/us/en/home/Products-and-Services/Services/Screening-and-Profiling-Services/SelectScreen-Profiling-Service/SelectScreen-Kinase-Profiling-Service.html

In the present study YA1 (a compound in accordance with formula (V)) was investigated for its ability to inhibit the activity of the 40 kinases shown in panel B of FIG. 2. The compounds of the invention were diluted in DMSO at a concentration of 10 mM and single-point kinase inhibitory activities were measured at 10 μM and Km ATP concentration.

### 1.2 Results

### 1.2.1 Compounds of the Invention Inhibit ROCK

The results of this assay are shown in FIG. 2A. These indicate that YA1, YA3 and YA4 had significantly less activity against ROCK than did their structural analogue Y27632. In contrast, YA2 exhibited ROCK inhibitory activity that was comparable to Y27632 (although further studies, reported below, indicated that this compound was least effective at preventing GEF16 colony formation).

### 1.2.2 Compounds of the Invention Inhibit a Range of Kinases

YA1, YA3 and YA4 are preferred compounds in accordance with the invention respectively representing compounds in accordance with formulas (V), (VII) and (VI).

Of these compounds, YA1 exhibited the greatest ability to inhibit GEF16 transformed colonies (see results discussed below and reported in FIG. 2A), and so may be considered a preferred compound in accordance with the present. An in vitro kinase inhibitory assay (SelectScreen™) was carried out in order to assess the ability of this compound to inhibit the activities of a representative selection of 40 human kinases, and the results of this assay are shown in (FIG. 2B).

The experimental data show that at 10 μM, YA1 had maximal inhibitory activity against p38 alpha (MAPK14) (72%), HGK (MAP4K4) (63%) and Aurora A (44%) and also confirmed the reduced ROCK inhibitory activity of YA1 shown in FIG. 2A (˜40%).

In light of these results, an additional single point analysis was carried out to investigate the inhibitory activity of each of the compounds of the invention (YA1, YA2, YA3 and YA4) against the kinases shown to be particularly effectively inhibited by YA1 (i.e. p38, HGK, Aurora A) and ROCK1 and ROCK2. The inhibitory activity of each compound was tested at a concentration of 10 μM.

The results obtained are illustrated in FIG. 2B and confirmed that YA2 had the greatest activity against ROCK's 1 and 2. In comparison, YA1, YA3 and YA4 all have significant activity against p38, HGK and Aurora A but show less activity against ROCKs than YA2.

### Study 2 Compounds of the Invention Inhibit Growth of Transformed Cells

It is known that Y27632, a structural analogue of compounds of the invention that shares these compounds' ability to inhibit kinase activity, is able to inhibit the growth of transformed cells in vitro. The following study was conducted to investigate the ability of compounds of the invention to inhibit growth of transformed cells.

### 2.1 Materials and Methods

### 2.1.1 Cell Culture and Stable Gene Transfection

The NIH3T3 mouse fibroblast cell line, was cultured in DMEM containing 10% bovine serum (BS) supplemented with 2 mM L-glutamine and grown at 37° C. in humidified air containing 5% CO₂.

Transformed cell populations were produced by transfection with constructs causing cellular expression of GEF16 or Ras as follows. The full-length GEF16 open reading frame (Accession NM_{—}014448) was PCR amplified, sequence verified and subcloned into the mammalian expression vector pCMVTag (Invitrogen Ltd., Paisley, UK) to produce a construct pCMVTag-GEF16 cDNA. This pCMVTag-GEF16 cDNA construct, or a construct causing Ras expression (the construct LZR-MS-IRES-ZEO/pBR-Ras), was then used to transfect NIH3T3 cells using Lipofectamine according to the manufacturer's recommendations (Invitrogen Ltd., Paisley, UK).

GEF16, Ras and vector control transfected cells were maintained in the presence of G418 or Zeocin for 10 days. Polyclonal GEF16, Ras and vector transfectants were expanded in sub-confluent cultures and −80° C. freezer stocks taken. Individual GEF16 transformed colonies were isolated by the use of cloning rings, expanded in culture and −80° C. frozen stocks also taken for storage.

Expression of GEF16 by in the transformed and control cell populations was investigated using RT-PCR, with beta-actin acting as a control. Total cellular RNAs were prepared using the SuperScript™ III Cells Direct cDNA Synthesis Kit as recommended by the manufacturer (Ambion, Cambridgeshire, UK), and total RNAs from samples were isolated using Trizol Reagent (Invitrogen Ltd., Paisley, UK). All DNAase I treated RNAs were then reverse-transcribed with random decamers. PCR was performed in 20 μl of a reaction mixture containing 2 μl of reverse-transcribed product, 10 μA of 2×Bio-Red and 0.1 μM of each primer. The specific primers for GEF16 and Beta-actin were as follows:

GEF16 forward
(Sequence ID No. 1)
5-ACCACCACCTCTTCTCCAAC-3′,
GEF16 reverse
(Sequence ID No. 2)
5′-TCGTTGGAGCAGTAGGCGAT-3′
Beta-actin forward:
(Sequence ID No. 3)
5′-TCC ATC ATG AAG TGT GAC GT-3′
Beta-actin reverse:
(Sequence ID No. 4)
5′-TCA GGA GGA GCA ATG ATC TT-3′

The reaction mixture was denatured at 94° C. for 4 min then amplified for 32 cycles of 30 sec denaturation at 94° C., 30 sec annealing at 55° C., and 30 sec extension at 72° C., followed by a single 5 min extension at 72° C.

### 2.1.2 Transformed Colony Forming Assay

Polyclonal vector or GEF16 transfected NIH3T3 cells, produced as described above, were seeded separately in 30 mm dishes at a density of 2×10⁵ cells per well and grown to full confluence in the presence of either 10 μM Y27632 (Calbiochem, Darmstadt, Germany), compounds of the invention (YA1, YA2, YA3, YA4) that are structural analogues of Y27632, or DMSO control. Medium (containing the compound of interest—either Y27632 or the selected compounds of the invention—or DMSO control) was changed every 2 days. Formation of foci was analysed by Toluidine blue (Sigma-Aldrich, Poole, UK) staining after 10 days growth post confluence. Toluidine blue staining reveals colonies as “dark” patches where staining is more intense than in other areas where colonies are absent. Each assay was carried out in triplicate and the results shown are representative of at least three separate experiments.

### 2.1.3 Cell Proliferation Assay

Cell proliferation was investigated using Celltiter Aq⁹⁶ reagent (Promega, Southampton, UK) according to the manufacturer's protocol. Cells were seeded into a 96-well plate at a density of 1×10³ cells/well allowing 3 wells per data point and allowed to attach for a set period. Following this, the initial starting point 490 nm absorbance was determined by adding 20 μl of Aq⁹⁶ reagent to each well and incubating for 4 h at 37° C. in 5% CO₂ (96-well plate reader, Dynex Technologies, West Sussex, UK). The various compounds (Y27632 or compounds of the invention) or DMSO control were then added to duplicate wells and the absorbance determined in the same way at the time points indicated. Each data set shown is representative of three separate experiments.

### 2.1.4 Flow Cytometry.

NIH3T3 cells with or without drug treatments (with Y27632 or the compounds of the invention) were harvested at various time points and cell counts carried out to confirm that 1×10⁶ cells were present for each cytometric analysis. Cells were washed with PBS, fixed with 70% ice-cold ethanol, pelleted and DNA stained by incubating the cells with propidium iodide (10 mg/mL) (Sigma-Aldrich, Poole, UK) at 4° C. for 45 min. Cells were then washed twice with ice-cold PBS and resuspended in 400 μl of PBS. The DNA content during different phases of the cell cycle was then determined by flow cytometry (BD Biosciences, Oxford, UK). Each profile shown was representative of three separate experiments.

### 2.2 Results

### 2.2.1 Constitutive Expression of GEF16 Transforms NIH3T3 Cells.

The results of RT-PCR are shown in FIG. 1A, and clearly illustrate that GEF16 mRNA was present in much greater quantities in cells transfected with the GEF16 vector, rather than those receiving the control vector.

It is known that NIH3T3 cells can be transformed by either ectopic expression of constitutively activated RhoA or various other guanidine exchange factors. The results obtained in the present study are consistent with these prior art data, and show (in FIG. 1A) that constitutive expression of GEF16 mRNA induces the formation of multiple transformed foci in NIH3T3 cells after 12 days of growth in the presence of G418. Multilayered transformed G418-resistant colonies were picked for further analysis and no transformed foci were observed in G418 selected vector transfected control cells. Comparison of the growth of vector and GEF16 transfected cells shows that there is no significant difference in proliferation rates between these two cell types (FIG. 1B, p>0.05).

### 2.2.2 Y27632 Inhibits the Formation of GEF16 Transformed Colonies.

Treatment of transformed cell colonies with 10 μM of Y27632 (a known inhibitor of ROCK) for 10 days suppresses the formation of GEF16 transformed NIH3T3 cell colonies (FIG. 1C), consistent with data previously reported in the prior art. FIG. 1D also illustrates that Y27632 inhibits the growth of confluent GEF16 transformed cells, yet these cells continue to proliferate in identical untreated cultures.

### 2.2.3 Compounds of the Invention Inhibit the Growth of GEF16 Transformed Colonies.

Y27632 is a structural analogue of ATP and 64 different analogues of this inhibitor were synthesised with the intention of evaluating their ability to inhibit the formation of GEF16 transformed NIH3T3 foci. Of these 64 analogues, four comprised YA1, YA2, YA3 and YA4, all of which are compounds of the invention.

At 10 μM, none of the 64 compounds synthesised showed any appreciable growth inhibitory activity (data not shown), yet the four compounds of the invention exhibited the ability to suppress the formation of GEF16 transformed colonies growing in post confluent cultures (FIG. 2A). Data obtained using this assay thus illustrate that compounds of the invention (such as YA1) are able to block transformed GEF16 colony formation to an extent comparable to the inhibition achieved using Y27632.

2.2.4 Transient Exposure of GEF16 Cells to Either YA1 or Y27632 Eliminates Transformed Colony Forming Cells from Polyclonal GEF16 Cells.

Freshly plated GEF16 polyclonal cells were treated with 10 μM of YA1 for 2, 4, 6, 8, and 10 days respectively after which the compound was removed from the culture media and the cells maintained in normal media for a further 10 day chase period. This shows a progressive decrease in the number of transformed foci associated with increased time of exposure to the compound (FIG. 3A) and indicates that YA1, a compound of the invention in accordance with formula (V), not only inhibits the formation of transformed foci but, on withdrawal, also prevents transformed foci from reforming. Significantly, there is no detectable difference in growth rates of sub-confluent GEF16 polyclonal cells treated with either inhibitor YA1 or DMSO control (FIG. 3B) (P>0.05), and flow cytometry shows no evidence of alterations in cell cycle or the accumulation of an apoptotic sub G1 population (FIG. 3C). This indicates that the compounds of the invention are not directly cytotoxic, and do not achieve their effects on transformed cells through killing of these cells.

In FIG. 3A the number of cells per dish increases with each successive inhibitor-treatment time-interval prior to withdrawal of the compound. In order to remove this variable and to ensure that each 10 day chase period starts with the same number of inhibitor treated cells, GEF16 polyclonal cells were treated with either Y27632 or a preferred compound of the invention (YA1) for 2, 4, 6, 8, and 10 days. Following this treatment cells were detached, re-seeded at 2×10⁵ per well in 6-well plates, and then maintained in the absence of Y27631 or YA1 for a 10 day chase period. This approach ensures that cell populations are directly comparable in terms of the number of cells at the beginning of the chase period.

The data obtained are shown in FIG. 4A. This illustrates that both Y27632 and a compound of the invention (YA1) are able to suppress transformed focus formation, and that the suppression achieved correlates with the duration of exposure of the transformed cells to the compound in question. Collectively these observations are the first to demonstrate that transient treatment of transformed cells with Y27632 or with compounds of the invention is able to permanently suppress transformed colony formation, but achieves this effect without cell killing.

### 2.2.5 Compounds of the Invention Eliminate Pre-Formed Transformed Colonies From GEF16 Polyclonal Cells.

The results reported above illustrated that both Y27632 and a compound of the invention (in particular YA1) are able to prevent transformed colonies from forming and that this effect persists after treatment with the compound has ceased. In order to evaluate the effects of these compounds on pre-formed colonies, polyclonal GEF16 NIH3T3 transformed colonies were allowed to form for 10 days and then exposed to Y27632 or a compound of the invention (YA1) for 3 and 6 days. It can be seen that Y27632 has very little effect on pre-formed transformed colonies whereas YA1 causes a marked reduction in their numbers (FIG. 4B). Vector transfected polyclonal NIH3T3 cells are included as a control and show no difference between inhibitor treated and DMSO controls.

2.2.6 The Compounds of the Invention Exhibit Minimal Effects on Cell Populations Derived from Single GEF16 Transformed Colonies.

Single transformed colonies were picked from GEF16 polyclonal NIH3T3 cells and expanded according to the methods described above. Expanded cell populations were plated and a compound of the invention (YA1) added either immediately or after 10 days when the cells were post-confluent.

The results of this study are shown in FIG. 5A, where it can be seen that treatment with YA1 (a compound of the invention according to formula (V)) has a modest effect when added to low density cultures, such that these do not achieve the same saturation density as populations of cells treated with a control. However, addition of this compound of the invention to post-confluent cultures has no discernible effect when compared to controls. Flow cytometry illustrates that the compound of the invention (YA1) has no effect on the cell cycle and no apoptotic sub G1 population is seen in cultures treated either pre or post confluence (further indicating that the compounds of the invention do not achieve their effects through the killing of cells).

2.2.7 Compounds of the Invention Suppress the Growth of Monoclonal GEF16 and Polyclonal Ras Transformed Cells when Transformed Cells are Co-Cultured with Non-Transformed Cells.

Non-transformed vector cells mixed with decreasing numbers of cells expanded from single GEF16 transformed colonies were treated with the compound of the invention YA1, or with DMSO, and incubated for 10 days. It can be seen that treatment with YA1 causes a marked reduction in the final saturation density of the cultures and that this is dependent on the number of transformed cells plated (FIG. 5B).

Comparing the results shown in FIG. 5B with those shown in FIG. 5A indicates that YA1 is more effective at suppressing the growth of transformed cells when these are in contact with non-transformed cells. FIG. 5C shows that YA1 produces the same growth suppressive effects on Ras transformed NIH3T3 cell colonies as with GEF16 transformed cells, and that these effects are also dependent on contact of the transformed cells with non-transformed cells. The phase contrast images also show that YA1 treated Ras transformed cells regain both contact inhibition and polarity.

### Study 3 Investigation of Gap Junction Formation in Response to Treatment with the Compounds of the Invention

The observation that the ability of compounds of the invention to suppress growth of transformed cells is influenced by contact of the transformed cells with non-transformed cells led the inventors to investigate the effects of the compounds of the invention on gap junction formation.

### 3.1 Materials and Methods

### 3.1.1 Quantitative Analysis of Gap Junction Formation.

A novel flow cytometric assay was developed to measure the extent of gap junction intercellular communication (GJIC) using two differentially stained cell populations. Non-transformed recipient NIH3T3-vector cells were stained with PKH67 (Excitation 490, Emission 502, Sigma-Aldrich, Poole, UK) and GEF16 transformed donor cells were stained with Lucifer yellow (LY) (Excitation 427 nm, Emission 517 nm, Invitrogen Ltd., Paisley, UK) as follows.

A 1 ml suspension of 1×10⁷ recipient cells in serum-free DMEM was mixed with an equal volume of 4 μM PKH67 solution and incubated for 5 min at room temperature. The reaction was terminated by adding 2 ml serum and incubating for 1 min. Cells were then washed three times with culture medium, and seeded at 1.5×10⁶ cells/T-25 flask.

For LY staining of transformed donor cells, 700 it of 5×10⁶ cells were mixed with 100 μl of 8 mg/ml LY solution in a 4 mm gap electroporation cuvette (EquiBIO, Middlesex, UK) and this was kept on ice for 5 min followed by electroporation at 400 V (1000 V/cm) (Gene Transformer™, Savant Instruments Inc., NY, USA). Fresh medium was added, the cells seeded in a T25 flask and allowed to recover overnight at 37° C.

LY labelled donor cells were then harvested and 1×10⁵ were added to the T25 flask containing the PKH67 labelled recipient cells plus 10 μM YA1 or DMSO control. After incubating for various time intervals, the co-cultured donor and recipient cells were collected and analysed using a BD FACS Aria™ (BD Biosciences, Oxford, UK). A 405 nm laser was used for LY excitation and emission was measured using a 515 nm to 545 nm band pass filter. GJIC between co-cultured donor and recipient cells was quantified as the percentage of LY and PKH67 double labelled cells.

### 3.2 Results

### 3.2.1 YA1 Increases Intercellular GJIC Between Transformed and Non-Transformed Cells.

FIG. 6A shows the effect of either YA1 or DMSO control on the extent of dye transfer from co-cultured donor LY labelled GEF16 single colony transformed cells to recipient PKH67 stained non-transformed cells. It can be seen from FIG. 6B that YA1 treated cultures have approximately 3 times the number of double LY/PKH67 labelled cells when compared to DMSO control (p<0.05). This indicates an increase in the transfer of dye from LY to PKH67 stained cells, which is consistent with an inhibitor induced increase in GJIC.

### 4 Statistical Analysis

All data referred to in the various studies presented above are from single or paired-experiments carried out in triplicate, or from 2-3 separate experiments in duplicate. Comparisons between groups were performed using paired or un-paired two-tailed Student's t-test. Statistical significance was taken to represent a p value<0.05.

### Discussion of the Results

The data provided above are the first to show that transient treatment with either the ROCK inhibitor Y27632 or with compounds of the invention (which are structural analogues of this compound) not only prevents the formation of transformed NIH3T3 colonies, but that this effect persists such that colonies do not form when the compounds are withdrawn. Most surprising was that, unlike Y27632, the effects of compounds of the invention in accordance with formula (V), formula (VI) or formula (VII) appear to be independent of ROCK inhibitory activity and do not involve cell killing. Furthermore, YA1 (a compound in accordance with formula (V) has the additional property of eliminating pre-existing transformed colonies and this effect is also not produced by cell killing.

Three of the compounds of the invention synthesised (YA1, YA3 and YA4) were initially shown to have similar effects to Y27632 on transformed colony formation yet these compounds had lower ROCK inhibitory activity. Paradoxically, although YA2 had equivalent ROCK inhibitory activity to Y27632, it was much less effective at preventing transformed colony formation.

The effects of Y27632 on transformed cells may not arise entirely due to ROCK inhibition. For example, in addition to ROCK's 1 and 2 it is known that at 10 μM Y27632 also has significant inhibitory activity against sixteen other kinases including the protein kinase C (PKC) isoforms beta, epsilon and eta, and the myotonic dystrophy kinase-related Cdc42-binding kinases Cdc42 BPA (MRCKA) and Cdc42 BPB (MRCKB). The Cdc42 activated MRCK kinases are particularly relevant since, like ROCKs, they promote myosin dependent cell motility and indicate a point of convergence between RhoA and Cdc42 signalling. Thus the observed inability of compound YA2 to prevent the formation of transformed colonies could be due to its lack of inhibitory activity against one or more of these alternative target kinases. Indeed PKC epsilon undergoes 94% inhibition by 10 μM Y27632 and it has been shown that increased activity of this kinase is causally associated with calpain inhibitor induced transformation of NIH3T3 cells. In addition, GEF16 possesses a potential Cdc42 binding motif and the inventors have demonstrated that GEF16 specifically activates Cdc42 in vitro and in cells. Interestingly, activated Cdc42 is also known to promote the activation of p38 which is entirely consistent with the p38 inhibitory activity of compounds YA1, YA3 and YA4.

The data presented in FIG. 2 suggest that the biological activity of YA1 may arise not through the inhibition of a single kinase target, but through the inhibition of a combination of targets, which may include an, as yet, unidentified kinase.

The observed minimal toxicity combined with persistence of the suppressive effects of the compounds of the invention on transformed colony formation, prompted an investigation into the rationale behind this effect. It has previously been suggested that the ability of non-transformed cells to establish GJIC with transformed cells serves to suppress the transformed properties of cells without cell killing.

The inventors hypothesised that inhibitor-induced increased GJIC between transformed and non-transformed NIH3T3 cells may be a highly plausible mode of action of the compounds of the invention. In order to address this issue the inventors developed the LY/PKH67 vital dye staining method described above. Since LY cannot penetrate cell membranes and PKH67 remains very stably associated with labelled cells the presence of cells double-labelled for both LY and PKH67 indicates that such cells are intercellularly linked. The results reported above clearly show that YA1 (a compound of the invention in accordance with formula (V)) induces increased accumulation of double LY/PKH67 labelled cells when compared to DMSO treated controls. This strongly supports a YA1 mediated increase in GJIC between transformed and non-transformed cells.

This suggestion is supported by the finding that treatment of single colony derived GEF16 transformed NIH3T3 cells with either YA1 or Y27632 has little or no effect when these are grown in the absence of non-transformed cells (results shown in FIG. 5A). However the growth inhibitory effects of YA1 on single colony derived transformed cells is clearly restored when these are co-cultured with non-transformed NIH3T3 cells (FIG. 5B). A surprising and unexpected result was the ability of this compound of the invention to eliminate pre-formed transformed colonies from GEF16 polyclonal cells (FIG. 5). Neither Y27632 nor the other compounds of the invention tested (data not shown) exhibited this property. This suggests that YA1 may have an additional mode of action that distinguishes it from the others compounds assayed.

The prior art provides supporting evidence to suggest that the various kinase targets described may participate in transformation induced disruption of gap junctions. For example, inhibition of ROCK activation by Y27632 has been shown to facilitate the formation of gap junctions in corneal epithelium, while H-Ras induced disruption of gap junctions in rat liver epithelial cells can be reversed by treatment with the p38 inhibitor SB203580. Further studies by the inventors found that SB203580 had some activity against GEF16 transformed colonies although this was much less than the compound of the invention YA1. Considering that 10 μM SB203580 has >20 fold more p38 inhibitory activity than YA1 at the same concentration (Tocris Biosciences, Data Sheet) this implies that p38 inhibition alone does not explain the inhibitory activity of YA1 against transformed colonies.

The inventors believe that it is very significant that YA1 was also highly effective at preventing the formation of Ras transformed NIH3T3 colonies, which indicates that the activity of these compounds is not restricted to a specific GEF but instead targets Rho/Ras mediated transformation in general.

In summary the data provided here support the hypothesis that specific inhibitors with the ability to modulate the activity of selected kinases may form the basis of a novel strategy for cancer chemo-prevention. The effect is most likely produced by enhancement of the ability of non-transformed cells to establish GJIC with transformed cells and it can clearly persist after withdrawal of the inhibitor. This finding suggests that treatments using the compounds of the invention as the active agent may not need to be administered continuously. Furthermore, the compounds of the invention may represent suitable agents for suppressing both the formation and growth of metastases. Y27632 (a structural analogue of the compounds of the invention) has been shown to suppress the development of metastases in vivo, and it has been suggested that this activity may arise through inhibition of ROCK suppressing the migration of tumour cells. The data provided here suggest an additional or alternative mode of action of Y27632 or the compounds of the invention through the promotion of intercellular communication between metastatic cells and normal cells at distant sites of invasion. This could provide an additional level of growth control during the metastatic process and thus allow the development of new therapies taking advantage of these properties.

### Synthetic Methods

### Preparation of Compound (V)

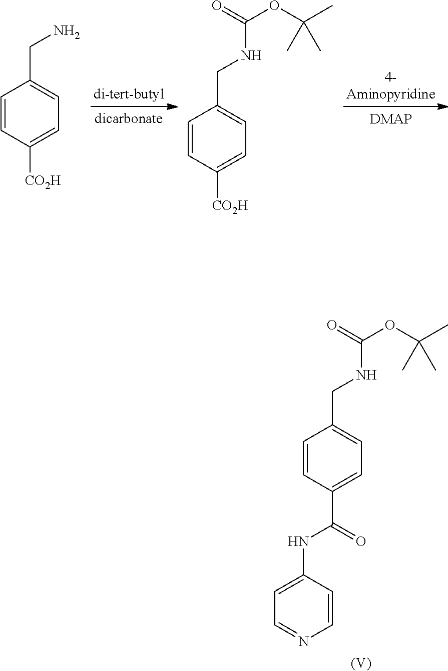

### Preparation of 4-[(tert-Butoxycarbonyl)aminomethyl]benzoic acid

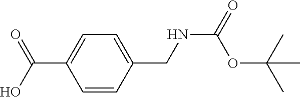 [SMILES: CC(C)(C)OC(=O)NCc1ccc(C(=O)O)cc1]

To a solution of commercially available 4-aminomethylbenzoic acid (10.078 g, 66.7 mmol, 1 eq.) in aqueous NaOH (5.866 g, 146.6 mmol, 2.2 eq.) in water (25 ml) and THF (tetrahydrofuran; 50 ml) was added di-tert-butyl dicarbonate (16 g, 73.3 mmol, 1.1 eq.). After stirring for 12 h the mixture was washed with hexane (2×50 ml), the aqueous phase cooled to 5° C. and adjusted to pH3 with aqueous saturated citric acid. The resulting white precipitate was extracted with ethyl acetate (3×50 ml) and the organic extracts combined and dried (MgSO₄). Concentration in vacuo yielded the title carbamate as white needles (14.43 g, 86%) from EtOAc.

R_{f}=0.43 (SiO₂ petrol: EtOAc; 1:3); νₘₐₓ (neat)/cm⁻¹ 3357 (m), 2968 (w), 2930 (w), 2884 (w), 2488 (m), 1682 (s), 1510 (m), 1409 (m), 1291 (m), 1244 (m), 1172 (m), 944 (m), 879 (m), 783 (m); δ_{H} (DMSO-d₆) 1.39 (9H, s, 3×CH₃), 4.18 (2H, d, J=6.3, CH₂), 7.34 (2H, d, J=8.1, H-3, 5), 7.48 (1H, t, J=6.1, NH), 7.89 (2H, d, J=8.2, H-2, 6), 12.87 (1H, s, br, COOH); δ_{C} (DMSO-d₆) 28.2 (3×CH₃), 43.2 (CH₂), 77.9 (C(CH₃)₃), 126.9 (CH), 129.2 (C-1), 129.3 (CH), 145.3 (C-4), 155.8 (C═O), carbamate), 167.2 (C═O, acid).

### Preparation of 4-(Pyridin-4-ylcarbamoyl)benzylcarbamic acid tert-butyl ester (compound (V))

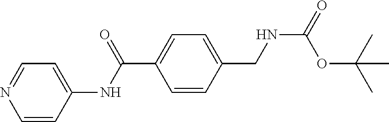 [SMILES: CC(C)(C)OC(=O)NCc1ccc(C(=O)Nc2ccncc2)cc1]

A solution of 4-[(tert-butoxycarbonyl)aminomethyl]benzoic acid (500 mg, 2 mmol, 1 eq.), EDC (1-ethyl-3-(3-dimethylaminopropyl) carbodiimide; HCl salt, 422 mg, 2.2 mmol, 1.1 eq.), DMAP (4-Dimethylaminopyridine; 24 mg, 0.2 mmol, 0.1 eq.) and 4-aminopyridine (188 mg, 2 mmol, 1 eq.) in dry dichloromethane (200 ml) was stirred overnight at room temperature. The mixture was washed with water (300 ml), saturated aqueous sodium bicarbonate (300 ml), water (300 ml), saturated brine (300 ml) and dried (MgSO₄). Concentration in vacuo followed by column chromatography (EtOAc) yielded the title amide compound (V) as a white solid (413 mg, 1.26 mmol, 63%) from EtOAc.

R_{f}=0.15 (SiO₂ EtOAc); νₘₐₓ (neat)/cm⁻¹ 3311 (w, br), 2977 (w), 2933 (w), 1684 (s), 1593 (s), 1521 (s), 1508 (s), 1331 (m), 1289 (m), 1167 (m), 828 (w); δ_{H} (DMSO-d₆) 1.40 (9H, s, 3×CH₃), 4.21 (2H, d, J=6.2, CH₂), 7.40 (2H, d, J=8.2, H-2, 6), 7.51 (1H, t, J=6.0, NH, carbamate), 7.78 (2H, dd, J=1.4, 4.9, H-3′, 5′), 7.92 (2H, d, J=8.2, H-3, 5), 8.47 (2H, d, J=5.9, H-2′, 6′), 10.54 (1H, s, NH, amide); δ_{c} (DMSO-d₆) 28.2 (3×CH₃), 43.2 (CH₂), 78.0 (C(CH₃)₃), 114.0 (C-3′, 5′), 126.8 (2×CH), 127.9 (2×CH), 132.6 (C-4), 144.6 (C), 146.0 (C), 150.3 (C-2′, 6′), 155.8 (C═O, carbamate), 166.3 (0=0, amide).

### Preparation of Compound (VI)

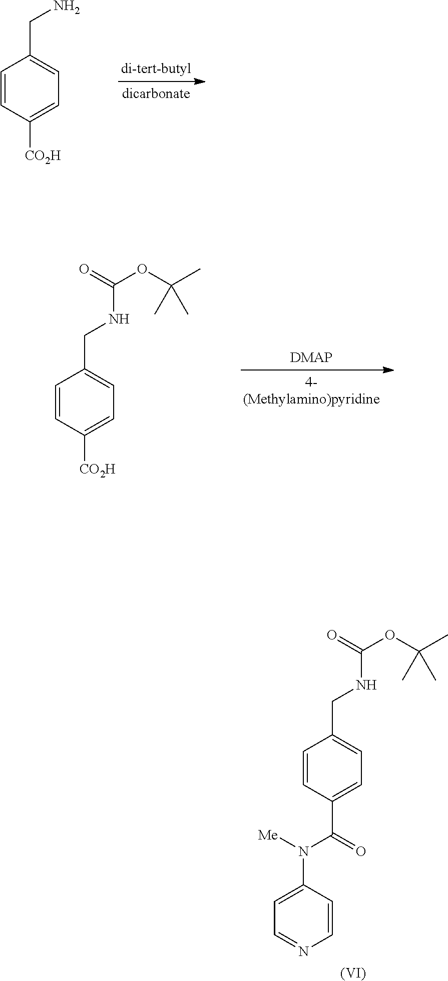

### Preparation of 4-[(tert-Butoxycarbonyl)aminomethyl]benzoic acid

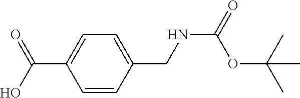 [SMILES: CC(C)(C)OC(=O)NCc1ccc(C(=O)O)cc1]

To a solution of commercially available 4-aminomethylbenzoic acid (10.078 g, 66.7 mmol, 1 eq.) in aqueous NaOH (5.866 g, 146.6 mmol, 2.2 eq.) in water (25 ml) and THF (Tetrahydrofuran; 50 ml) was added di-tert-butyl dicarbonate (16 g, 73.3 mmol, 1.1 eq.). After stirring for 12 h the mixture was washed with hexane (2×50 ml), the aqueous phase cooled to 5° C. and adjusted to pH3 with aqueous saturated citric acid. The resulting white precipitate was extracted with ethyl acetate (3×50 ml) and the organic extracts combined and dried (MgSO₄). Concentration in vacuo yielded the title carbamate as white needles (14.43 g, 86%) from EtOAc.

R_{f}=0.43 (SiO₂ petrol: EtOAc; 1:3); νₘₐₓ (neat)/cm⁻¹ 3357 (m), 2968 (w), 2930 (w), 2884 (w), 2488 (m), 1682 (s), 1510 (m), 1409 (m), 1291 (m), 1244 (m), 1172 (m), 944 (m), 879 (m), 783 (m); δ_{H} (DMSO-d₆) 1.39 (9H, s, 3×CH₃), 4.18 (2H, d, J=6.3, CH₂), 7.34 (2H, d, J=8.1, H-3, 5), 7.48 (1H, t, J=6.1, NH), 7.89 (2H, d, J=8.2, H-2, 6), 12.87 (1H, s, br, COOH); δ_{C} (DMSO-d₆) 28.2 (3×CH₃), 43.2 (CH₂), 77.9 (C(CH₃)₃), 126.9 (CH), 129.2 (C-1), 129.3 (CH), 145.3 (C-4), 155.8 (C═O), carbamate), 167.2 (C═O, acid).

### Preparation of 4-(N-Methyl-N-pyridin-4-ylcarbamoyl)benzylcarbamic acid tert-butyl ester (Compound (VI))

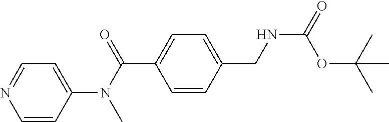 [SMILES: CN(C(=O)c1ccc(CNC(=O)OC(C)(C)C)cc1)c1ccncc1]

The title amide compound (VI) was prepared from 4-[(tert-butoxycarbonyl)aminomethyl]benzoic acid (500 mg, 2 mmol, 1 eq.), EDC (1-ethyl-3-(3-dimethylaminopropyl) carbodiimide; HCl salt, 422 mg, 2.2 mmol, 1.1 eq.), DMAP (4-Dimethylaminopyridine; 24 mg, 0.2 mmol, 0.1 eq.) and 4-(methylamino)pyridine (215 mg, 2 mmol, 1 eq.) following the procedure for compound (V) set out above. Purification by column chromatography (petrol: EtOAc; 1:9) yielded the title amide compound (VI) as white crystals (495 mg, 1.45 mmol, 73%) from EtOAc.

R_{f}=0.11 (SiO₂ petrol: EtOAc; 1:9); νₘₐₓ (neat)/cm⁻¹ 3342 (m, br), 3033 (w), 2978 (m), 2931 (w), 2247 (w), 1707 (s), 1653 (s), 1589 (s), 1500 (m), 1365 (s), 1169 (s), 731 (m); δ_{H} (CDCl₃): 1.43 (9H, s, 3×CH₃), 3.50 (3H, s, CH₃), 4.26 (2H, d, J=5.6, CH₂), 4.82 (1H, s, NH, carbamate), 6.91 (2H, dd, J=1.6, 4.6, H-3′, 5′), 7.14 (2H, d, J=8.2, H-2, 6), 7.26-7.32 (2H, m, H-3, 5), 8.42 (2H, dd, J=1.6, 4.6, H-2′, 6′); δ_{C} (CDCl₃): 28.6 (3×CH₃), 37.6 (CH₃), 44.4 (CH₂), 80.0 (C(CH₃)₃), 120.4 (C-3′, 5′), 127.3 (C-3, 5), 129.3 (C-2, 6), 134.1 (C-4), 142.1 (C-1), 150.9 (C-2′, 6′), 152.2 (C-4′), 156.0 (C═O, carbamate), 170.6 (C═O, amide).

### Preparation of Compound (VII)

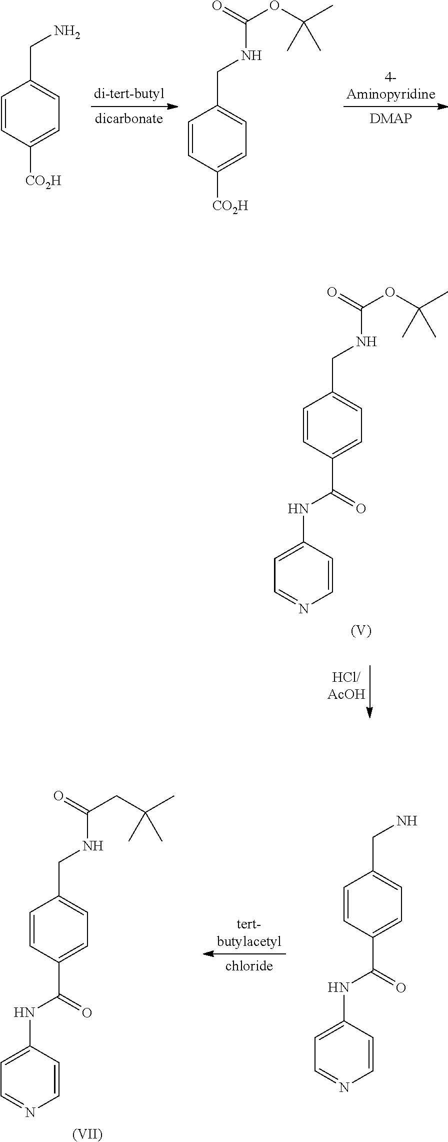

Compound (V) was prepared in two steps as set out above.

### Preparation of 4-(Aminomethyl)-N-pyridin-4-ylbenzamide

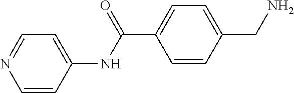 [SMILES: NCc1ccc(C(=O)Nc2ccncc2)cc1]

A solution of 4-(pyridin-4-ylcarbamoyl)benzylcarbamic acid tert-butyl ester (3.62 g, 11.06 mmol, 1 eq.) in 1M HCl in acetic acid (20 ml, 20 mmol) was stirred for 1 h. After adding ether (200 ml) the resulting white precipitate was filtered off and recrystallised from water as a white powder (1.68 g, 7.8 mmol, 71%).

R_{f}=0.00 (SiO₂ EtOAc); νₘₐₓ (neat)/cm⁻¹ 3352 (w), 1664 (s), 1591 (s), 1506 (m), 1417 (m), 1332 (m), 1289 (w), 1211 (w), 823 (m), 532 (w); δ_{H} (DMSO-d₆) 2.16 (2H, s, NH₂), 3.80 (2H, s, CH₂), 7.50 (2H, d, J=8.5, H-2, 6), 7.79 (2H, dd, J=1.6, 4.8, H-3′, 5′), 7.92 (2H, d, J=8.4, H-3, 5), 8.47 (2H, dd, J=1.6, 4.8, H-2′, 6′), 10.52 (1H, s, NH); δ_{C} (DMSO-d₆) 45.3 (CH₂), 114.0 (C-3′, 5′), 126.9 (2×CH), 127.8 (2×CH), 132.0 (C-1), 146.0 (C), 148.8 (C), 150.3 (C-2′, 6′), 166.3 (C═O).

### Preparation of 4-[(3,3-Dimethylbutylcarbonyl)aminomethyl]-N-pyridin-4-ylbenzamide (Compound (VII))

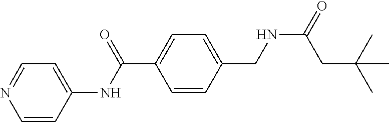 [SMILES: CC(C)(C)CC(=O)NCc1ccc(C(=O)Nc2ccncc2)cc1]

A solution of 4-(aminomethyl)-N-pyridin-4-ylbenzamide (227 mg, 1.0 mol, 1 eq.), tert-butylacetyl chloride (153 μl, 1.1 mmol, 1.1 eq.) and pyridine (160 μl, 2 mmol, 2 eq) in DCM (Dichloromethane; 5 ml) was stirred overnight at ambient. The mixture was washed with sat. aq. NaHCO₃ (10 ml), brine (10 ml), dried (MgSO₄) and concentrated in vacuo. Purification by column chromatography (EtOAc) yielded the title amide compound (VII) as a white solid (216 mg, 0.66 mmol, 66%) from EtOAc.

R_{f}=0.08 (SiO₂ EtOAc); m.p. 187.5° C.; νₘₐₓ (neat)/cm⁻¹ 3296 (w), 3034 (w), 2979 (w), 2927 (w), 1685 (s), 1655 (s), 1590 (s), 1519 (s), 1270 (m), 1145 (w), 1108 (w), 817 (w), 529 (w); δ_{H} (DMSO-d₆) 0.97 (9H, s, 3×CH₃), 2.05 (2H, s, CH₂CO), 4.34 (2H, d, J=6.0, CH₂N), 7.42 (2H, d, J=8.3, H-3, 5), 7.78 (2H, dd, J=1.6, 4.8, H-3′, 5′), 7.93 (2H, d, J=8.3, H-2, 6), 8.37 (1H, t, J=6.1, NH-bn), 8.47 (2H, dd, J=1.4, 4.9, H-2′, 6′), 10.54 (1H, s, NH-py); δ_{C} (DMSO-d₆) 29.7 (3×CH₃), 30.5 (C(CH₃)₃), 41.7 (CH₂N), 48.7 (CH₂CO), 114.0 (C-3′, 5′), 127.2 (2×CH), 127.9 (2×CH), 132.5 (C-1), 144.4 (C), 145.9 (C), 150.3 (C-2′, 6′), 166.2 (C═O), 170.9 (C═O).

1. A compound having the formula (I) 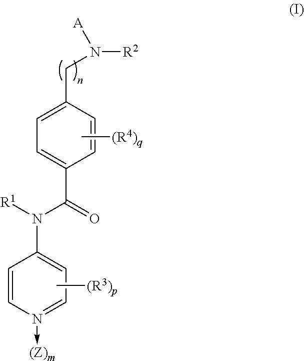
wherein: 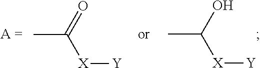
X is oxygen or methylene; Y is C₃₋₆ alkyl or aryl; Z is oxygen or C₁₋₃ alkyl;
R¹ is hydrogen or C₁₋₃ alkyl; R² is hydrogen or C₁₋₃ alkyl; the or each R³ is separately C₁₋₃ alkyl or halo; the or each R⁴ is separately C₁₋₃ alkyl or halo; p is 0 to 4; q is 0 to 4; m is 0 or 1; and n is 1 to 3.

2. A compound according to claim 1, wherein A has the formula

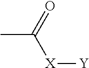

3. A compound according to claim 1, wherein p and/or q is 0.

4. A compound according to claim 1, wherein p is 1 to 4 and the or each R³ is selected from the group consisting of methyl, ethyl, n-propyl and i-propyl, any of which may be substituted or unsubstituted, and/or is selected from the group consisting of fluoro, chloro, bromo and iodo.

5. A compound according to claim 1, wherein q is 1 to 4 and the or each R⁴ is selected from the group consisting of methyl, ethyl, n-propyl and i-propyl, any of which may be substituted or unsubstituted, and/or is selected from the group consisting of fluoro, chloro, bromo and iodo.

6. A compound according to claim 1, wherein R² is hydrogen.

7. A compound according to claim 1, wherein R² is selected from the group consisting of methyl, ethyl, n-propyl and i-propyl, any of which may be substituted or unsubstituted.

8. A compound according to claim 1, wherein m is 0.

9. A compound according to claim 1, wherein m is 1 and Z is oxygen, or Z is methyl, ethyl, n-propyl and i-propyl, any of which may be substituted or unsubstituted.

10. A compound according to claim 1, wherein n is 1.

11. A compound according to claim 1, wherein n is 2 or 3.

12. A compound according to claim 1, wherein Y is a substituted or unsubstituted, linear, branched or cyclic, optionally unsaturated group C₃₋₆ alkyl group selected from the group consisting of propyl, butyl, pentyl and hexyl.

13. A compound according to claim 1, wherein Y is selected from the group consisting of t-butyl and —C(CF₃)₃.

14. A compound according to claim 1, wherein Y is a substituted or unsubstituted aryl group.

15. A compound according to claim 1, wherein X is oxygen.

16. A compound according to claim 1, wherein R¹ is selected from the group consisting of hydrogen and a methyl group.

17. A compound according to claim 1, wherein the compound has a formula selected from the group consisting of:

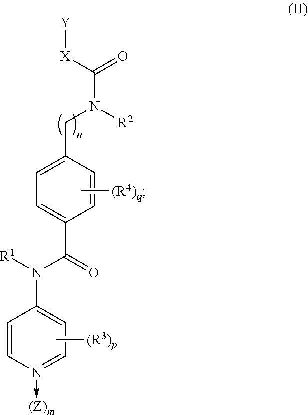

18-19. (canceled)

20. A compound selected from the group consisting of:

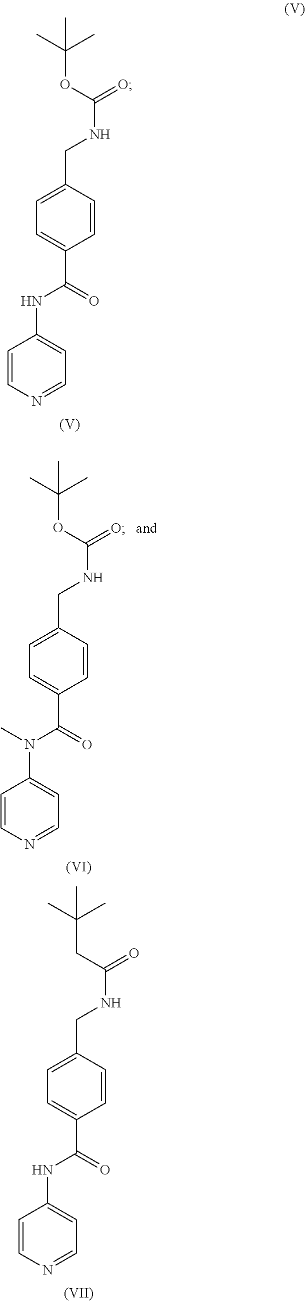

21-22. (canceled)

23. A compound of formula (I) for use as a medicament.

24. A compound according to claim 23 for use in chemotherapy.

25. A compound according to claim 23 for use in the prevention and/or treatment of pain or inflammation or of a condition associated with pain or inflammation.

26. A compound of formula (I) for use as a medicament for the prevention and/or treatment of cancer.
